(19) United States
(12) Plant Patent Application Publication
(10) Pub. No.: US 2010/0071104 P1
(43) Pub. Date: Mar. 18, 2010

(54) Lomandra hystrix plant named 'LHBYF'

(50) ;

(76) Inventor: Todd Anthony Layt, Richmond (AU)

Correspondence Address: Sam R. McCoy Jr., P.O. Box 2108, Mount Pleasant, SC, 29465, US

(21) Appl. No.: 12/284,007

(22) Filed: Sep. 17, 2008

(51) Int. Cl. A01H 5/00

(52) U.S. Cl. PLT/384

(57) ABSTRACT

‘LHBYF’ is a distinctive variety of Lomandra hystrix which is characterized by the combination of its tall, medium density plant growth habit with long leaves and large, prominent inflorescences with many flowers borne in spring and summer.

### LATIN NAME OF THE GENUS AND SPECIES

The Latin name of the genus and species of the novel variety disclosed herein is Lomandra hystrix.

### VARIETY DENOMINATION

The inventive variety of Lomandra disclosed herein has been given the variety denomination ‘LHBYF’.

### BACKGROUND OF THE INVENTION

The present invention relates to a new and distinct perennial variety of Lomandra hystrix, which has been given the variety denomination of ‘LHBYF’. Its market class is that of an ornamental grass-like plant. ‘LHBYF’ is intended for use in landscaping and as a decorative grass-like plant.

The Lomandra hystrix variety ‘LHBYF’ was first discovered in 1997 in an Australian nursery in the state of New South Wales during a routine inspection of approximately 3000 seedlings of open pollinated Lomandra hystrix (unpatented) production stock. ‘LHBYF’ is a seedling selection of these open pollinated Lomandra hystrix. Initially 50 plants were selected due to their growth vigor. These were grown on and further observed and then the three with the best male flowers were selected and planted into a garden for further evaluation. Finally in late 2003 a single plant was selected due to its prolific flowering borne on a large inflorescence combined with vigorous growth. It was named ‘LHBYF’.

‘LHBYF’ was first propagated asexually by division in the state of New South Wales, Australia and has since been asexually propagated by division and micropropagation. The distinctive characteristics of the inventive ‘LHBYF’ variety are stable from generation to generation; clones of the variety produced by asexual reproduction maintain the distinguishing characteristics of the original plant.

‘LHBYF’ has a dense growth habit, a tall plant height and leaves that are typical of common Lomandra hystrix (unpatented in the United States).

An application for plant breeders' rights for variety ‘LHBYF’ has been lodged with the Australian Plant Breeders' Rights Office, and was accepted on Oct. 26, 2006 under Application No. 2006/270.

### SUMMARY OF THE INVENTION

‘LHBYF’ is a distinctive variety of Lomandra hystrix which is characterized by the combination of its dense plant growth habit, tall plant height and large inflorescence size with many flowers.

### BRIEF DESCRIPTION OF THE FIGURES

FIG. 1 shows a ‘LHBYF’ plant.

FIG. 2 shows ‘LHBYF’ flower detail

### BOTANICAL DESCRIPTION OF THE PLANT

The following is a detailed botanical description of a new and distinct variety of a Lomandra hystrix ornamental plant known as ‘LHBYF’. Plant observations were made on plants grown in New South Wales, Australia. Unless indicated otherwise, the descriptions disclosed herein are based upon observations made in December 2006 of mature ‘LHBYF’ plants grown in nursery pots.

Those skilled in the art will appreciate that certain characteristics will vary with older or, conversely, younger plants. ‘LHBYF’ has not been observed under all possible environmental conditions. Where dimensions, sizes, colors and other characteristics are given, it is to be understood that such characteristics are approximations or averages set forth as accurately as practicable. The phenotype of the variety may vary with variations in the environment such as season, temperature, light intensity, day length, cultural conditions and the like. Color notations are based on The Royal Horticultural Society Colour Chart, The Royal Horticultural Society, London, 1995 edition. ‘LHBYF’ has not flowered to date. ‘LHBYF’ is undergoing further trialing and comparative testing in Australia and the United States.

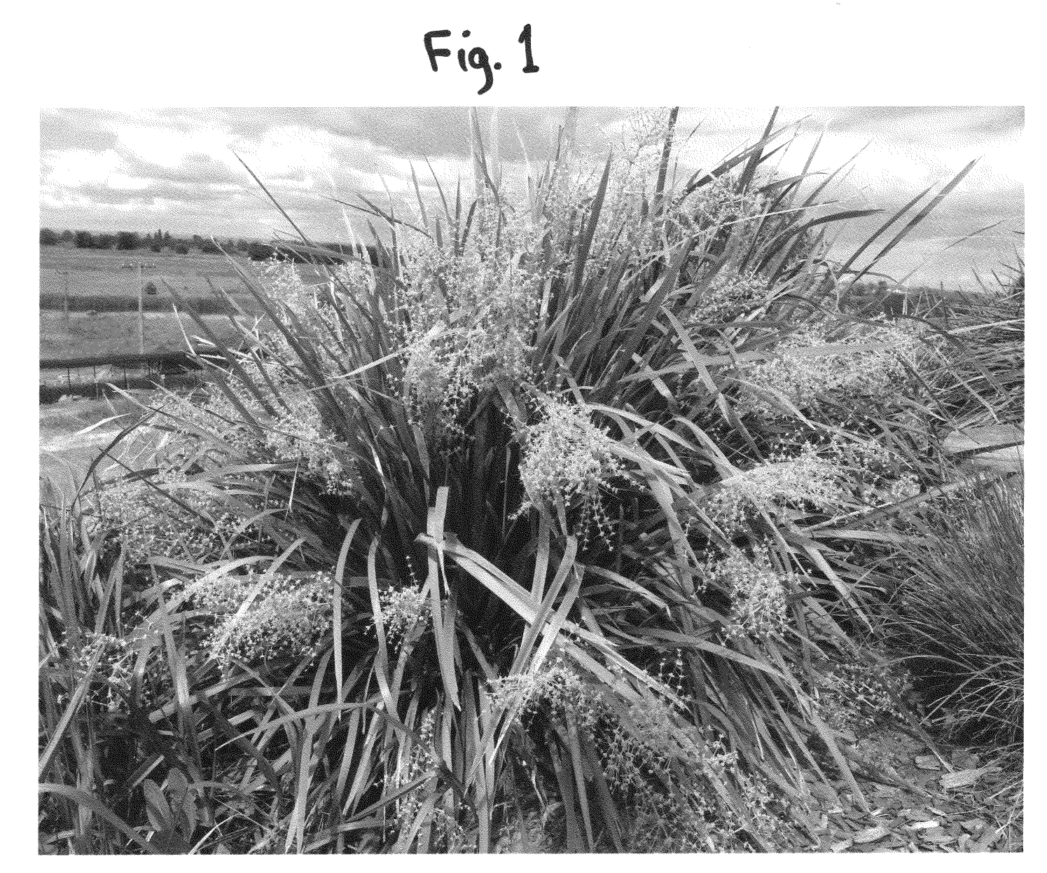

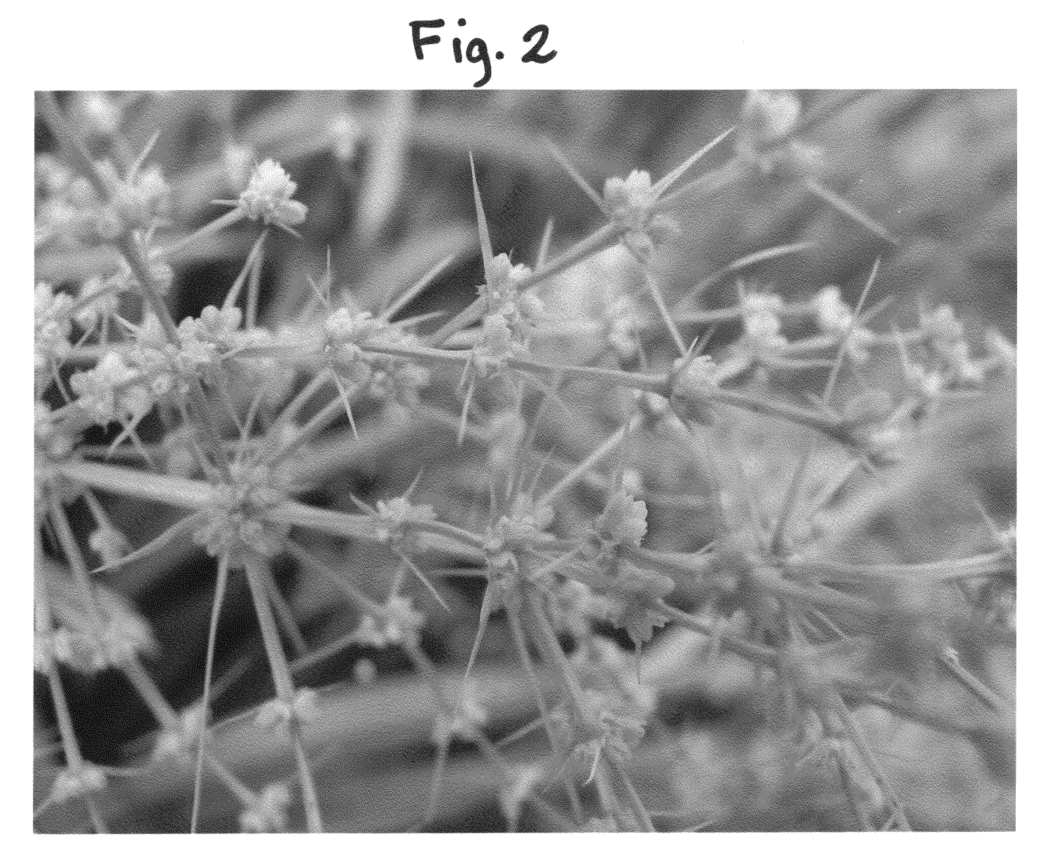

‘LHBYF’ is a perennial Lomandra hystrix plant which originated from a seedling selected from a population of open pollinated plants of Lomandra hystrix. After its selection, ‘LHBYF’ was asexually propagated by division and subsequently by micropropagation. ‘LHBYF’ has a medium leaf blade width combined with a densely shooted plant growth habit, a tall plant height and a large inflorescence size with a large number of flowers giving a prominent flowering display which is unusual for Lomandra hystrix plants which usually have a medium inflorescence size and a less prominent flowering display.
Growth habit, dimensions and color: ‘LHBYF’ is a tall, rhizomatous plant forming a dense tussock. Plant height is 80-90 cm and plant spread is 110-130 cm in a mature plant grown in a 300 mm nursery or field pot in Sydney, New South Wales, Australia. The upper and lower side of the leaf is yellow green (RHS 146A) in color. Variegation is absent. The leaf blade is generally 13 to 16 mm in width, and 70-90 cm in length. The leaf base is cauline, leaf margin is entire, leaf apex is tridentate and leaf surface glaucosity is weak to absent. The leaf venation pattern is parallel; the color is the same as the rest of the leaf (yellow-green RHS 146A; observed summer 2007). The leaf is generally a uniform width from base to the tip, margins may shred at the base, with a tridentate apex typical of Lomandra hystrix. The leaf apex may become locally necrotic in response to moisture stress. The basal leaf margin color prior to any shredding consists of brown RHS 200D. A representative ‘LHBYF’ plant is shown in FIG. 1.
Roots: Similar to other Lomandra hystrix, ‘LHBYF’ has a large root structure. The roots are fibrous and spreading, similar to other Lomandra hystrix.
Rhizomes: Short (3 cm to 4 cm); rhizome color with the leaf sheath removed is white (RHS 155C); surface texture of the rhizome is smooth.
Inflorescence: ‘LHBYF’ has a large inflorescence size with a large number of flowers giving a prominent flowering display. Each inflorescence is visible from within the foliage and emerges to display to above the foliage. This is unusual for Lomandra hystrix plants which usually have a medium inflorescence size and a less prominent flowering display. The inflorescence is a panicle with single branching and usually 4 branches per node. Flowers are male and borne in whorled clusters around each node. Each branch of the panicle is subtended by a bract of length 10-14 cm near the base of the peduncle and reducing in length to 1-2 cm towards the apex of the inflorescence. Each cluster of flowers is subtended by 4 bracts of length 10-20 mm. Inflorescence height is from 75-85 cm and inflorescence width is from 22-30 cm. The peduncle is flattened in profile and peduncle color is approximately yellow-green RHS 146A-B. FIG. 1 shows a ‘LHBYF’ plant demonstrating the prominence of the inflorescences.
Flowers: Flowers are male and borne in clusters around inflorescence nodes. Tepal color is a dull yellow corresponding approximately to RHS 10A-B. Flower length is 3-4 mm usually. FIG. 2 shows ‘LHBYF’ flower detail.
Flower rachis: The length of the flower rachis (the branch of the panicle) is from 40 mm towards the apex to 150 mm towards the middle and base of the inflorescence (observed summer 2006-2007).
Reproductive organs of male florets: Six stamens, anther size is 0.5 mm to 0.8 mm in length, anther color is yellow (approximately RHS 8B), observed summer 2006-2007.
Seed: Flowers are male; seed is not produced.
Flowering period: Flowering season is from spring to summer. ‘LHBYF’ is undergoing further testing.
Lastingness of bloom: Approximately 14 days in summer flowering in Sydney, New South Wales, Australia.
Fragrance: Fragrance is typical of the species, and of medium strength.
Comparison of ‘LHBYF’ with other varieties of Lomandra hystrix: ‘LHBYF’ has a taller plant height than “LHCOM’, another variety of L. hystrix with a more compact growth habit. ‘LHBYF’ has more upright basal shoots which creates a more conical shaped tussock whereas ‘LHCOM’ has a horizontal basal shoot attitude that produces a more rounded tussock. ‘LHBYF’ has a medium plant density whereas ‘LHCOM’ has a dense plant density. The leaf blade length of ‘LHBYF’ is longer (70-90 cm) than that of ‘LHCOM’ (60-65 cm). The leaf blade width of ‘LHBYF’ is broader (13-16 mm) than that of ‘LHCOM’ (11-12 mm). ‘LHBYF’ has large, prominent inflorescences with many flowers from spring to summer whereas ‘LHCOM’ has not flowered thus far. In comparison with the common form of L. hystrix, ‘LHCOM’ is more floriferous with larger, more prominent inflorescences with many flowers on tall, wide single branching panicles. Flowers on ‘LHCOM’ are male only whereas the common form of L. hystrix has both female and male flowers borne on separate plants. The combination of its tall, medium density plant growth habit with long leaves and large, prominent inflorescences with many flowers makes ‘LHBYF’ an attractive ornamental grass-like plant.
Asexual reproduction: After its initial discovery, ‘LHBYF’ was transplanted into a garden bed for further trials and testing. After divisions were made for a second generation, ‘LHBYF’ was observed to retain plant growth characteristics that were noted in the original ‘LHBYF’ seedling. ‘LHBYF’ was then divided into many larger pots for further evaluation and introduction to micropropagation.
Environmental tolerances: ‘LHBYF’ has shown potential for shade tolerance and further shade tolerance tests are underway. The winter hardiness of ‘LHBYF’ is at least to zone 8a in the Southeastern United States, and evaluation of winter hardiness is ongoing. ‘LHBYF’ has been observed to hold color to −10 degrees Celsius without any noticeable change in appearance of the plant. ‘LHBYF’ has excellent drought tolerance. After severe wilting, ‘LHBYF’ has been noted to recover with watering. ‘LHBYF’ does well in sandy soils, but also tolerates heavy, clay-type soils well.
Disease resistance: ‘LHBYF’ has good resistance to root rot compared with most other Lomandra hystrix and evaluation of disease resistance is ongoing.

That which is claimed is:

1. A new and distinct variety of Lomandra hystrix plant named ‘LHBYF’, substantially as described and illustrated herein.
